(19) United States
(12) Patent Application Publication
(10) Pub. No.: US 2015/0376698 A1
(43) Pub. Date: Dec. 31, 2015

(54) METHOD FOR ESTABLISHING THE SOURCE OF INFECTION IN A CASE OF FEVER OF UNCLEAR AETIOLOGY

(71) Applicants: Stefan Russwurm, Jena (DE); Konrad Reinhart, Jena (DE); Michael Bauer, Jena (DE)

(72) Inventors: Stefan Russwurm, Jena (DE); Konrad Reinhart, Jena (DE); Michael Bauer, Jena (DE)

(21) Appl. No.: 14/711,720

(22) Filed: May 13, 2015

Related U.S. Application Data
(63) Continuation of application No. 12/305,195, filed on Oct. 20, 2009, now abandoned.

(51) Int. Cl. C12Q 1/68

(52) U.S. Cl. CPC C12Q 1/6883; C12Q 2600/158; C12Q 2600/136; C12Q 2600/106

(57) ABSTRACT

Use of gene expression profiles obtained in vitro from a patient's sample for establishing the local infection of a “fever of unknown origin”, wherein the gene expression profiles are specific for local inflammations of a “fever of unknown origin”, such as peritonitis, pneumonia, endocarditis or infections of the urea tract.

### CROSS-REFERENCES

This application claims priority under 35 U.S.C. 120 to U.S. patent application Ser. No. 12/305,195 filed Oct. 20, 2009, a U.S. National Stage application under 35 U.S.C. 371 of international application no. PCT/EP2007/005043 filed on Jun. 6, 2007 which claims priority from German patent application no. DE 10 2006 027 842.9 filed on Jun. 16, 2006, all of which are hereby incorporated by reference in their entirety. All references noted herein are also hereby incorporated by reference.

### DESCRIPTION

The present invention relates to the use of gene expression profiles obtained in vitro from a patient's sample for establishing the local inflammation of a fever of unknown origin according to claim 1, a method for measuring in vitro such gene expression profiles according to claim 14, as well as the use of the gene expression profiles and/or of the probes used therefore for establishing the gene activity or the protein products derived therefrom for the screening of active agents against fever of unknown origin and/or peritonitis and/or pneumonia and/or the evaluation of the therapeutic effects of active agents against fever of unknown origin and/or peritonitis and/or pneumonia according to claim 30, as well as a kit according to claim 33.

Fever of unknown origin (FUO) clinically is defined as a fever with a temperature of more than 38.8° C. lasting over a period of more than 3 weeks, wherein no clear diagnosis regarding the origin could be made after one week of examination. Depending on the origin, there are four classes of FUO described: FUO of classical, nosocomial, immune deficient, or HIV-related origin (1). FUO also was described as rather a known disease with an unusual clinical picture than a rare deficiency (2).

There is neither a gold standard method nor a diagnosis test, there are no published regulations and no evidence based recommendations for the diagnosis of FUO (3). Up to now, the diagnosis of FUO is a challenge and it is made with the aid of the patient's history, of biopsies (e.g. liver, temporal artery), surgical and/or imaging methods such as abdominal computer tomography or nuclear spin imaging methods (3). All these methods are very expensive and unpleasant for the patient (1) because of the surgical intervention (biopsy, surgery). The following 4 subgroups can be defined with regard to the diagnosed main cause: Infections, malignant tumor, autoimmune disorders and other causes, wherein infection is the most frequent cause of FUO (1, 4).

An infection was recorded in only 10% of the patients suffering from post operative fever (5). In most cases, the temperature of the patient returned to normal within four days after the surgical intervention. In spite of this fact, some patients developed an infection on the fifth day after the surgery and 12% of them fell ill to pneumonia (5). Similarly, Pile and his colleagues mentioned that fever occurring two days after the surgery was highly likely triggered by an infection such as, for example, an infection of the urinary tract and/or the inner abdomen (peritonitis), pneumonia, an infection triggered by an intravenous catheter.

Different forms, such as peritonitis, pneumonia, infections of the urea tract or endocarditis (2), can be the local inflammation conditions underlying the FUO. In the following, peritonitis and pneumonia are described, by way of example only, as the inflammation condition underlying FUO.

In an intensive care unit, pneumonia is one of the most severe infectious diseases which may have dramatic effects on the patient's life expectancy (6,7). Pneumonia is an acute or chronic inflammation of the lung parenchyma, which is mostly caused by an infection by bacteria, viruses or fungi. For clinical diagnostics, a difference is made between pneumonia caught in ambulant or nosocomial treatment. 2-3 million cases of pneumonia caused in ambulant treatment were registered in the USA, whereas experts assume that 750,000 cases of ambulant acquired pneumonia occurred in Germany (8). The costs for pneumonia treatment in the USA alone mount up to approx. US$ 8 bn.

Pneumonia is defined as being nosocomial if the pneumonia is diagnosed 48 hours after admission of the patient into the hospital (9). The greatest risk of development of a nosocomially acquired pneumonia in patients in intensive care is caused by the use of ventilators. For this reason, the term ventilator associated pneumonia (VAP) became known for this kind of pneumonia (10). The mortality rate in VAP patients is 30% (10).

According to Sauer et al., only 30% of the infections triggered by individual pathogens could be proven in the course of a study of infections caused by surgical operations. According to Sauer, the most common cause of infection in pneumonia was candida (yeast). In patients suffering from pneumonia, mixed infections with at least two kinds of pathogens (47%), one single pathogen (24%) or no microbes at all (29%) were identified. A possible infection and the resistance is determined on the basis of conventional microbiologic methods of cultivation as well as on resistance tests towards antibiotics (11) and, therefore, underlies the limitations of such methods (non-culturable bacteria, an extended retardation phase due to the administration of antibiotics, etc.).

Peritonitis is a local infection of the peritoneum caused by the entry of bacteria or fungi into the abdominal cavity. Peritoneal mesothelial cells (PMC) in the muscular part of the membrane are interrupted by intermesothelial gaps (stomata) and thus render the contact with the cavities (lacunae) in the lymphatic vessel and the exit of bacteria from the abdominal cavity (12) possible. According_{—} to Hall et al., the quick removal of bacteria from the abdominal cavity is an explanation for the initial septic phase of a peritonitis. An infection of the abdominal cavity is dealt with by means of three different mechanisms: 1. Induction of immune defense such as, for example, the release of inflammation mediators, 2. the migration of polymorphonuclear neutrophiles and the complement cascade, and 3. the formation of an abscess.

Usually, peritonitis involves mixed microbial populations (12), however, the outcome of a peritonitis varies depending on the pathogen that has caused the peritonitis (13). Troidle et al., for example, describe that Gram-negative infections lead to a higher mortality and that these patients are more likely to need a hospital stay than in the case of Gram-positive pathogens. In the case of Gram-positive peritonitis, a re-occurrence of the infection at a later time takes place in 32% of the cases, whereas, in comparison, this rate is 9% in the case of Gram-negative peritonitis (9%). In spite of many publications which show the effects of the pathogens on the patient (for example 12), some authors asses the reaction of the host to an infection more important than the infection itself (14). These assessments established from animal models, however, base on a physiologic evaluation system and do not use genomic or proteomic experiments.

New biomolecular methods allow the analysis of the immunologic host response to an infection. Different methods and results are known from the state of the art describing the differential gene activity as response to an disease caused by an infection (15-19).

The basic usability of gene expression profiles which, for example, can be obtained by means of the micro array technology, for the diagnosis of SIRS, generalized inflammatory inflammations, sepsis and severe sepsis, is described in the PCT application of the Applicant of the present invention (20) or (21), which is herein incorporated by reference.

The German patent application (22) shows for the first time gene activity marker for the differentiation between infectious and non-infectious multiple organ failure. This application describes the use of 1,297 different genes for in vitro diagnosis of patients suffering from infectious and non-infectious multiple organ failure, respectively.

It was also possible to show different organ specific studies regarding differential gene expression caused by local inflammations, such as by the examination of lung tissue (19, 23-25) or by examination of changed gene activity of liver tissue in response to faecal peritonitis (26). The tests, however, always related to tissue-specific changes in gene activity, and are, thus, not suitable for establishing a FUO by means of measurement of the gene activity in body fluids.

In the patent application (27) the gene expression is used for establishing the infectious and non-infectious condition of the indentified source of infection and it is not used for determining the source of infection. In order to determine, for example, whether there exists an infection in the knee joint, a biopsy is carried out and the cells contained in the synovial fluid are analyzed. This invention does not teach the examination of the differential gene activity in body fluids for establishing the underlying local inflammation of a FUO.

Both Reinhart et al. (28) and the not yet prepublished German patent application (29) of the Applicant of the present invention (28), presented gene expression profiles obtained from whole blood of patients in which SIRS and Sepsis, respectively, were diagnosed. The differential gene activity was used in order to evaluate whether gene activity classificators can differentiate between infectious and non-infectious inflammatory diseases. In this study, the experimentally ascertained gene activity classificators were subsequently compared to the clinical parameters available from the patients. It was shown that the identified gene activity classificators are able to well differentiate between infectious and non-infectious conditions if the clinical data pointed to a peritonitis as underlying local inflammation. The ability to differentiate between infectious and non-infectious conditions, however, was reduced when the clinical data indicated a ventilator-associated pneumonia (VAP). The gene activity classificators described by Reinart (2005) and in reference 29, respectively, thus allow the differentiation between infectious and non-infectious conditions. A possibility to establish the underlying local condition of a FUO by means of gene expression profiles was neither disclosed nor rendered obvious.

Thus, there is urgent need for possibilities for in vitro diagnosis of the underlying local inflammation in a fever of unknown origin. The availability of such in vitro methods will render the diagnosis of FUO quick and not as painful for the patient, allow for appropriate therapeutic measures, and significantly reduce the costs of the treatment.

The origin of the invention disclosed in the present patent application is the realization that gene activity profiles can be used to determine the underlying local inflammation of a FUO. The use of these gene activities is not possible with the clinical parameters conventionally used for diagnosis, however, it is very important for the initiation of a specialized therapy in intensive care.

Thus, it is the object of the present invention to use gene activity markers in order to make it possible to establish the local inflammation of a fever of unknown origin.

This object is solved by the features of claims 1, 14 and 33.

The present invention relates in particular to the use of gene expression profiles that have been obtained in vitro from a patient's sample for the establishment of the local inflammation of a fever of unknown origin.

A preferred embodiment of the present invention relates to the use of specific gene expression profiles which permit die localization of the underlying local inflammations. Examples for said local inflammations of a FUO are peritonitis, pneumonia, endocarditis or infections of the urinary tract.

The invention in particular relates to the gene expression profiles of at least 2 polynucleotides, selected from SEQ-IDs No 1 to 191, which are specific for peritonitis or pneumonia as local inflammations of a “fever of unknown origin”. Here, the gene activities of the polynucleotides with SEQ-IDs No 1 to 191 having similar expression activities can be pooled into diagnostic gene activity clusters.

These gene activity cluster are composed as follows:

Cluster 1: SEQ-ID No. 1 to SEQ-ID No. 77 peritonitis specific sequences with significant gene activity (table 3)

Cluster 2: SEQ-ID NO. 78 to SEQ-ID No. 191 pneumonia specific sequences with significant gene activity (table 3)

The invention furthermore comprises gene expression profiles of at least 2 polynucleotides, selected from SEQ-ID No. 192 to SEQ-ID No. 432, which are specific for a local inflammation, but not for peritonitis or pneumonia, of a “fever of unknown origin”.

Another embodiment of the invention also comprises gene expression profiles of at least 2 polynucleotides comprising 80% homology to SEQ-IDs No. 1 to SEQ-ID No. 432, for establishing the local inflammation of a fever of unknown origin.

The invention also includes the use of these gene expression profiles as inclusion or exclusion criterion to decide whether patients suffering from “fever of unknown origin” are included in clinical studies.

Another embodiment of the invention is the use of the gene expression profiles obtained in vitro for the creation of gene activity data for electronic further processing. These gene activity data can be used for the production of software for the description of the individual prognosis of a patient, for diagnosis purposes and/or patient data management systems.

Another use of the gene expression profiles obtained in vitro is the preparation of clinical expert systems and/or the modeling of cellular signal transduction pathways. Like modeling methods and/or programs are, for example, ingenuity (Fa. Ingenuity Systems), Panther (Applied Biosystems) or other methods known to the person skilled in the art.

A preferred embodiment is characterized in that a specific gene and/or gene fragment is used for the generation of gene expression profiles, the gene and/or gene fragment being selected from a group consisting of SEQ-ID No. 1 to SEQ-ID No. 432 as well as gene fragments thereof with at least 20-2000 nucleotides.

A further embodiment of the invention is characterized in that the gene fragments comprise 20-200, preferably 20-80, nucleotides.

A further embodiment of the invention is characterized in that the gene expression profiles are determined by means of hybridization methods, in particular hybridization methods basing on micro arrays or real-time PCR. Hybridizing methods are well known to the person skilled in the art.

One further embodiment of the invention is a method, characterized in that for in vitro measurement of gene expression profiles and/or at least one gene activity cluster for establishing a local inflammation of a fever of unknown origin, characterized in that—in patients—the gene activity of a plurality of predetermined genes related to the source of infection are determined in a patient's sample.

Another embodiment of the invention is characterized in that for in vitro measurement of gene expression profiles and/or at least one gene activity cluster for establishing peritonitis or pneumonia as source of infection of a fever of unknown origin, in patients, the gene activity of a plurality of predetermined genes related to peritonitis and pneumonia as source of infection are determined in a patient's sample, wherein the genes and/or gene fragments specific for peritonitis and pneumonia of the local inflammation are selected from the group consisting of: SEQ-ID No. 1 to SEQ-ID No. 191 as well as gene fragments therefrom with at least 20-2000 nucleotides.

Another embodiment of the invention is characterized in that the specific sequences SEQ-ID No. 1 to SEQ-ID No. 191 are composed of the following diagnostic clusters:
Cluster 1: SEQ-ID No. 1 to SEQ-ID No. 77 peritonitis specific sequences with significant gene activity
Cluster 2: SEQ-ID No. 78 to SEQ-ID No. 191 pneumonia specific sequences with significant gene activity

A further embodiment of the invention is characterized in that the gene fragments comprise 20-200, preferably 20-80 nucleotides.

Another embodiment of the present invention is characterized in that at least 4 to 100 different genes and gene fragments are used.

Another embodiment of the present invention is characterized in that at least 200 different genes and/or gene fragments are used.

Another embodiment of the present invention is characterized in that at least 200 to 500 different genes and/or gene fragments are used.

Another embodiment of the present invention is characterized in that at least 500 to 1000 different genes and gene fragments are used.

Another embodiment of the present invention is characterized in that at least 1000 to 2000 different genes and gene fragments are used.

Another embodiment of the invention is characterized in that the genes or gene fragments listed in table 3 and table 4 and/or the sequences derived from their RNA are replaced by: synthetic analogues, aptamers, Spiegelmers as well as peptido- and morpholinonucleic acids.

Another embodiment of the invention is characterized in that the synthetic analogues of the genes comprise 20-100, in particular approx. 70 base pairs.

Another embodiment of the present invention is characterized in that the gene activity is determined by means of hybridization methods.

Another embodiment of the present invention is characterized in that the gene activity is determined by means of microarrays.

Another embodiment of the invention is characterized in that the gene activity is determined by hybridization-independent methods, in particular by enzymatic and/or chemical hydrolysis and/or amplification methods, preferably PCR, subsequent quantification of nucleic acids and/or of derivates and/or fragments thereof.

Another embodiment of the present invention is characterized in that the sample is selected from: tissue, body fluids, in particular blood, serum, plasma, urine, saliva or a mixture thereof.

Another embodiment of the present invention is characterized in that samples, in particular cell samples, are subjected to a lytic treatment, in order to release their cell contents.

In another embodiment of the invention, gene expression profiles that are obtained in vitro from a patient's sample and/or of probes used therefore, selected from the group consisting of SEQ-ID No. 1 to SEQ-ID No. 191 as well as gene fragments thereof with at least 20-2000 nucleotides are used for determining the gene activity or the protein products derived therefrom for the screening of active agents against fever of unknown origin and/or peritonitis and/or pneumonia and/or for the evaluation of the therapeutic effects of active agents against fever of unknown origin and/or peritonitis and/or pneumonia.

Another embodiment of the invention is characterized in that hybridizable synthetic analogues of the probes listed in tables 3 and 4 are used.

A further embodiment of the invention is characterized in that the gene fragments comprise 20-200, preferably 20-80 nucleotides.

The invention also relates to a kit containing a selection of sequences which are specific for the establishment of the local inflammation of a “fever of unknown origin”, and/or gene fragments thereof with at least 20-2000 nucleotides for the determination of gene expression profiles in vitro in a patient's sample, for determining of a source of infection and/or the source of infection of a fever of unknown origin.

Another embodiment of the invention is characterized in that the kit contains a selection of at least 2 polynucleotides with sequences according to SEQ-ID No. 1 to SEQ-ID No. 191 and/or gene fragments thereof with at least 20-2000 nucleotides for determining gene expression profiles in vitro in a patient's sample, for establishing peritonitis and/or pneumonia as local inflammation of a fever of unknown origin.

### Working Example

Test for the creation of gene expression profiles to establish the local inflammation of patients diagnosed with fever of unknown origin (1,3) and severe infection (30).

### Measurement of the Differential Gene Expression

First of all, the differential gene expression between two groups of patients was tested, wherein the following was known from the groups:

i) the first (partially blinded) group were patients suffering from a severe infection [sepsis, classified according to 30] in the course of their intensive care treatment and diagnosed with “fever of unknown origin” (patient group 1). The local inflammation underlying the FUO was not known in these patients.
ii) the second group were patients who developed an acute generalized inflammation [SIRS, classified according to 30] with organ failure in the course of their treatment in intensive care, but in whom no infection was detected at any time during their treatment in intensive care (patient group 2).

Selected characteristics of both patient groups are shown in table 1. Information includes age, sex, as well as the SOFA-score as a measure for the function of the organ systems. In addition, the plasma protein levels of procalcitonine (PCT) and CRP as well as the number of leukocytes of the patients are given.

Reference samples were total RNA from SIG-M5 cell lines.

Each of the patients' samples was co-hybridized with the reference sample on one microarray each.

TABLE 1
Data of patient groups 1 and 2

| | patients with | | | |
|---|---|---|---|---|
| | severe infection | | SIRS + OD | |
| | patient group 1 | | patient group 2 | |
| Number of patients | 39 | | 37 | |
| Mortality | 16 | (41.0%) | 2° | (5.4%) |
| Sex [m/f] | 31/8 | | 18/19 | |
| Age [years] | 69 | (11) | 75 | (14) |
| SOFA Score | 9 | (2.5) | 8* | (2) |
| Number of OD | 3 | (1) | 2 | (1) |
| PCT [ng/ml] | 2.44 | (3.20) [36] | 3.34 | (4.13) [30] |
| CRP [mg/l] | 177 | (124.4) [35] | 91.6* | (90.13) [36] |
| WBC [no/l] | 14400 | (9050) | 11900* | (7400) |

median (IQR)
*p < 0.05 (Wilcoxon rang sum test)
°p = 0.003 (exact test of Fisher)

### Experimental Description:

### Drawing Blood and Isolation of RNA

At the time when “fever of unknown origin” was diagnosed, the whole blood of patient group 1 was drawn postoperatively from the patients by means of the PAXGene Kit according to the manufacturer's (Qiagen) instructions. The whole blood of patient group 2 was postoperatively drawn by means of the PAXGene Kit Kit according to the manufacturer's (Qiagen) instructions. After drawing whole blood, the total RNA of the samples was isolated using the PAXGene Blood RNA kit according to the manufacturer's (Qiagen) instructions.

### Cell Cultivation

For cell cultivation (control samples) 19 cryo cell cultures (SIGM5) (frozen in liquid nitrogen) were used. The cells were each inoculated with 2 ml Iscove's medium (Biochrom AG) supplemented with 20% fetal calf serum (FCS). Subsequently, the cell cultures were incubated in 12 well plates for 24 hours at 37° C. in 5% CO2. Subsequently, the content of the 18 wells was parted in 2 parts with the same volume each, so that finally 3 plates of the same format (36 wells in total) were available. Afterwards, the cultivation was continued under the same conditions for 24 hours. Afterwards, the resulting cultures of 11 wells of each plate were combined and centrifuged (1000×g, 5 min, ambient temperature). The supernatant was removed and the cell pellet was dissolved in 40 ml of the above mentioned medium. These 40 ml of dissolved cells were distributed in equal shares in two 250 ml flasks and again incubated after adding 5 ml of the above-mentioned medium. 80 μl of the remaining 2 ml of the two remaining plates were placed in empty wells of the same plates that had previously been prepared with 1 ml of the above-mentioned medium. After 48 hours of incubation, only one of the 12 well plates was processed as follows: 500 μl were extracted from each well and combined. The resulting 6 ml were introduced into a 250 ml flask comprising approximately 10 ml of fresh medium. This mixture was centrifuged for 5 minutes with 1000×g at ambient temperature and dissolved in 10 ml of the above-mentioned medium. The following results were obtained by subsequent counting of cells: 1.5×107 cells per ml, 10 ml total volume, total number of cells: 1.5×108. As the number of cells was not yet sufficient, 2.5 ml of the above-mentioned cell suspension was introduced into 30 ml of the above-mentioned medium in a 250 ml (75 cm²) flask (4 flasks in total). After 72 hours of incubation 20 ml of fresh medium were added to each flask. After the subsequent incubation of 24 hours, the cells were counted as described above. The total amount of cells was 3.8×10⁸ cells. In order to obtain the desired number of cells of 2×106 cells, the cells were resuspended in 47.5 ml of the above mentioned medium in 4 flasks. After the incubation time of 24 hours, the cells were centrifuged and washed two times with phosphate buffer in absence of Ca²⁺ and Mg²⁺ (Biochrom AG).

The isolation of the total RNA is performed by means of NucleoSpin RNA L Kits (Machery&Nagel) according to the manufacturer's instructions. The above described process was repeated until the necessary number of cells was obtained. This was necessary to obtain the necessary amount of 6 mg total RNA corresponding to an efficiency of 600 μg RNA per 108 cells.

### Reverse Transcription/Labeling/Hybridization

After drawing whole blood, the total RNA of the samples was isolated and tested for quality using the PAXGene Blood RNA kit (PreAnalytiX) according to the manufacturer's instructions. 10 μg total RNA were aliquoted from each sample and transcribed with 10 μg total RNA from SIGM5 cells as reference RNA to complementary DNA (cDNA) by means of the reverse transcriptase Superscript II (Invitrogen). Subsequently, the RNA was removed from the mixture by alkaline hydrolysis. In the reaction mixture a part of the dTTP was replaced by aminoallyl-dUTP (AA-dUTP) in order to render the linkage of the fluorescent dye to the cDNA possible at a later point of time.

After the purification of the reaction mixture, the cDNA of the samples and the controls were covalently labeled with the fluorescent dyes Alexa 647 and Alexa 555 and hybridized on a microarray of the SIRS-Lab company. On the microarray used, 5308 polynucleotides with lengths of 55 to 70 base pairs were immobilized. Each of the polynucleotides represents a human gene. Additionally there were control spots for quality assurance. One microarray is divided into 28 subarrays, each of the subarrays being arranged in a grid of 15×15 spots.

The hybridization and the subsequent washing and drying, respectively, were carried out according to the manufacturer's instructions for 10.5 hours at 42° C. using the hybridization station HS 400 (Tecan). The hybridization solution used was composed of the cDNA samples, each labelled, 3.5×SSC (1×SSC comprises 150 mM sodium chloride and 15 niM sodium citrate), 0.3% sodium lauryl sulfate (v/v) 25% formamide (v/v) and each 0.8 μl cot-1 DNA, yeast t-RNA and poly-A RNA. The subsequent washing of the microarrays was carried out at ambient temperature according to the following scheme: Rinse 90 seconds with washing buffer 1 (2×SSC, 0.03% sodium lauryl sulfate), with washing buffer 2 (1×SSC) and finally with washing buffer 3 (0.2×SSC). Subsequently, the microarrays were dried under a nitrogen flow at a pressure of 2.5 bar for more than 150 seconds at 30° C.

After hybridization, the hybridization signals of the microarrays were read by means of the GenePix 4000B (Axon) scanner and the expression ratios of the different expressed genes were determined by means of the GenePix Pro 4.0 (Axon) software.

### Evaluation:

For the analysis, the average intensity of one spot was determined as median value of the corresponding spot pixel.

### Correction of Systematic Errors:

Systematic errors were corrected according to the approach of Huber et al. [31]. According to this approach, the additive and the multiplicative bias in a microarray was estimated on the basis of 70% of the gene samples present. For all further computations, the signals were transformed by means of arcus sinus hyperbolicus.

For the analysis, the normalized and transformed relative ratios of the signals of the patients samples were calculated with respect to the general control. This means that the calculation for the gene no. j of the patient no. n revealed the data Gj,n=arc sin h(Scy5(j,n))−arc sin h(Scy3(j,n)), wherein [SCy3(j,n), SCy5(j,n)] is the associated signal pair. When a spot could not be analyzed for a patient (e.g. scanned picture is stained), the associated value was marked as “missing value”.

### Statistical Comparison:

For comparison the paired random student test was employed per gene. Both random tests contained the values of the patient groups. In order to select the differentially expressed genes, the corresponding p-value was evaluated. It applied for the group of the selected genes that the associated p-value was smaller than 0.05.

In the sequence listing attached to the present application, the sequences indicated in tables 3 and 4 are individually allocated to one sequence ID (Sequence ID: 1 to Sequence ID: 432). Thus, the gene activities ascertained and shown in tables 3 and 4 can be used for the distinction of infectious and non-infectious conditions. These results confirm the methods and results from the state of the art, as for example shown in (20-22).

Unblinding of Patient Group 1 and Correlation with the Ascertained Gene Activities of Table 3 and 4.

The unblinding of patient group 1 revealed that this patient group consisted of two subgroups:

1) Patients, in which FUO and a severe infection were diagnosed and the follow-up diagnosis identified peritonitis as underlying local inflammation (patient group 1a).
2) Patients, in which FUO and a severe infection were diagnosed and the follow-up diagnosis identified pneumonia as underlying local inflammation (patient group 1b).

Selected characteristics of the two patient groups 1a and 1b subsequent to the follow-up diagnosis are shown in table 2.

TABLE 2
Data of patient groups 1a and 1b

| | patient group 1 a | | patient group 1b | |
|---|---|---|---|---|
| Number of patients | 15 | | 24 | |
| Mortality | 9 | (60%) | 7 | (29.2%) |
| Sex [m/f] | 11/4 | | 20/4 | |
| Age [years] | 66 | (9) | 70 | (13) |
| SOFA Score | 9 | (2.5) | 9 | (2.25) |
| Number of OD | 3 | (0) | 2.5 | (1) |
| PCT [ng/ml] | 6.05 | (24.3) [13] | 1.46* | (1.98) [23] |
| CRP [mg/l] | 146 | (87.5) | 206 | (95.75) [20] |
| WBC [no/l] | 14400 | (11000) | 14650 | (6875) |

| | | |
|---|---|---|
| Local inflammation | peritonitis | pneumonia |

In order to establish a local inflammation underlying a FUO in patients, the determined gene activities from table 3 and 4 were statistically classified according to significant gene activity clusters which showed a similar activity within patient groups 1a and 1b. In this context, it was surprisingly found out that, basing on all gene activities measured, a classification of gene activities into three cluster resulted:

Cluster 1: For peritonitis, a cluster of specific sequences with significant gene activity according to SEQ-ID No. 1 to SEQ-ID No. 77 was determined, which are part of the enclosed sequence listing.

Cluster 2: For pneumonia, a cluster of specific sequences with significant gene activity corresponding to SEQ-ID No. 78 to SEQ-ID No. No. 191 was determined, which are part of the enclosed sequence listing.

Cluster 3: Common set of sequences with similar significant gene activity in patients with severe infections which are specific for a local inflammation, but not for peritonitis or pneumonia of a “fever of unknown origin”, corresponding to SEQ-ID No. 192 to SEQ-ID No. 432, which are part of the enclosed sequence listing.

The three gene activity cluster are shown in table 3 (cluster 1 and 2) and 4 (cluster 3).

TABLE 3
Gene activity cluster 1 and 2 for establishing peritonitis (cluster
1) or pneumonia (cluster 2) as local inlammation of a FUO

| Gen Bank | | normalized and transformed expression signals | | | | | |
|---|---|---|---|---|---|---|---|
| Accession No. | p value | mean patient group 2 | mean patient group 1a | UniGene | Cluster | subgroup | SeqID |
| AA029887 | 0.0003 | −0.145 | 0.174 | | 1 | Peritonitis | 1 |
| AA149226 | 0.0028 | −0.248 | 0.298 | Hs.494192 | 1 | Peritonitis | 2 |
| AA398757 | 0.0000 | −0.665 | 0.798 | Hs.634201 | 1 | Peritonitis | 3 |
| AA402274 | 0.0110 | −0.216 | 0.259 | Hs.567266 | 1 | Peritonitis | 4 |
| AA419092 | 0.0046 | 0.152 | −0.182 | Hs.122575 | 1 | Peritonitis | 5 |
| AA435854 | 0.0000 | 0.197 | −0.236 | | 1 | Peritonitis | 6 |
| AA441793 | 0.0081 | −0.159 | 0.191 | Hs.132753 | 1 | Peritonitis | 7 |
| AA458827 | 0.0002 | −0.42 | 0.503 | Hs.500546 | 1 | Peritonitis | 8 |
| AA479285 | 0.0296 | −0.094 | 0.113 | Hs.536450 | 1 | Peritonitis | 9 |
| AA490815 | 0.0073 | 0.144 | −0.173 | Hs.558393 | 1 | Peritonitis | 10 |
| AA620762 | 0.0272 | −0.14 | 0.168 | Hs.371845 | 1 | Peritonitis | 11 |
| AA629051 | 0.0017 | 0.13 | −0.156 | | 1 | Peritonitis | 12 |
| AA693514 | 0.0224 | 0.09 | −0.107 | Hs.134229 | 1 | Peritonitis | 13 |
| AA708806 | 0.0014 | −0.129 | 0.154 | Hs.596038 | 1 | Peritonitis | 14 |
| AA731679 | 0.0415 | −0.235 | 0.282 | Hs.128619 | 1 | Peritonitis | 15 |
| AI057616 | 0.0207 | −0.102 | 0.123 | Hs.83761 | 1 | Peritonitis | 16 |
| AI086719 | 0.0347 | 0.105 | −0.126 | Hs.127657 | 1 | Peritonitis | 17 |
| AI128170 | 0.0432 | 0.111 | −0.133 | Hs.592034 | 1 | Peritonitis | 18 |
| AI150418 | 0.0353 | 0.111 | −0.133 | Hs.545647 | 1 | Peritonitis | 19 |
| AI218498 | 0.0480 | 0.113 | −0.135 | Hs.585282 | 1 | Peritonitis | 20 |
| AI221860 | 0.0304 | −0.229 | 0.275 | Hs.208353 | 1 | Peritonitis | 21 |
| AI241294 | 0.0484 | 0.125 | −0.15 | Hs.308641 | 1 | Peritonitis | 22 |
| AI282924 | 0.0001 | −0.166 | 0.199 | Hs.591290 | 1 | Peritonitis | 23 |
| AI291041 | 0.0003 | 0.215 | −0.258 | | 1 | Peritonitis | 24 |
| AI374599 | 0.0015 | 0.132 | −0.158 | Hs.128060 | 1 | Peritonitis | 25 |
| AI418437 | 0.0066 | −0.264 | 0.317 | Hs.534383 | 1 | Peritonitis | 26 |
| AI420865 | 0.0035 | −0.148 | 0.178 | Hs.54190l | 1 | Peritonitis | 27 |
| AI499146 | 0.0126 | −0.147 | 0.176 | | 1 | Peritonitis | 28 |
| AI520932 | 0.0211 | −0.111 | 0.133 | Hs.516707 | 1 | Peritonitis | 29 |
| AI627453 | 0.0401 | 0.104 | −0.125 | Hs.370510 | 1 | Peritonitis | 30 |
| AI634473 | 0.0158 | 0.107 | −0.128 | Hs.603284 | 1 | Peritonitis | 31 |
| AI696984 | 0.0069 | −0.121 | 0.145 | Hs.262907 | 1 | Peritonitis | 32 |
| AI732971 | 0.0169 | 0.179 | −0.214 | Hs.559775 | 1 | Peritonitis | 33 |
| AI912592 | 0.0005 | −0.148 | 0.177 | Hs.86538 | 1 | Peritonitis | 34 |
| BC004983 | 0.0486 | −0.103 | 0.124 | Hs.81328 | 1 | Peritonitis | 35 |
| BC032713 | 0.0047 | −0.131 | 0.157 | Hs.87968 | 1 | Peritonitis | 36 |
| CARD10 | 0.0003 | −0.154 | 0.184 | Hs.57973 | 1 | Peritonitis | 37 |
| CCL15.2 | 0.0184 | −0.096 | 0.115 | Hs.272493 | 1 | Peritonitis | 38 |
| CCL26 | 0.0259 | 0.127 | −0.153 | Hs.131342 | 1 | Peritonitis | 39 |
| CCL27 | 0.0082 | 0.129 | −0.154 | Hs.459590 | 1 | Peritonitis | 40 |
| CR1 | 0.0001 | −0.167 | 0.2 | Hs.334019 | 1 | Peritonitis | 41 |
| DNAJB2 | 0.0000 | −0.226 | 0.271 | Hs.77768 | 1 | Peritonitis | 42 |
| FADD | 0.0004 | −0.153 | 0.184 | Hs.86131 | 1 | Peritonitis | 43 |
| GH1.1 | 0.0033 | −0.268 | 0.322 | Hs.406754 | 1 | Peritonitis | 44 |
| H05223 | 0.0000 | −0.175 | 0.21 | Hs.124638 | 1 | Peritonitis | 45 |
| H11661 | 0.0000 | −0.223 | 0.267 | Hs.504091 | 1 | Peritonitis | 46 |
| H91663 | 0.0009 | −0.151 | 0.181 | Hs.208052 | 1 | Peritonitis | 47 |
| IF | 0.0084 | −0.158 | 0.19 | Hs.312485 | 1 | Peritonitis | 48 |
| IL2IR | 0.0065 | −0.179 | 0.214 | Hs.210546 | 1 | Peritonitis | 49 |
| IL2RB | 0.0223 | 0.341 | −0.409 | Hs.474787 | 1 | Peritonitis | 50 |
| KBRAS2 | 0.0000 | −0.204 | 0.244 | Hs.632252 | 1 | Peritonitis | 51 |
| MAP2K2 | 0.0000 | −0.195 | 0.235 | Hs.465627 | 1 | Peritonitis | 52 |
| N64541 | 0.0088 | 0.172 | −0.207 | Hs.597199 | 1 | Peritonitis | 53 |
| NM_004710 | 0.0388 | −0.088 | 0.105 | Hs.464210 | 1 | Peritonitis | 54 |
| NM-001774 | 0.0002 | −0.192 | 0.23 | Hs.166556 | 1 | Peritonitis | 55 |
| NM-002649 | 0.0001 | −0.177 | 0.212 | Hs.602240 | 1 | Peritonitis | 56 |
| NM-006058 | 0.0018 | −0.208 | 0.25 | Hs.543850 | 1 | Peritonitis | 57 |
| NM-006732 | 0.0046 | −0.255 | 0.307 | Hs.590958 | 1 | Peritonitis | 58 |
| NM-014339 | 0.0019 | −0.146 | 0.175 | Hs.129751 | 1 | Peritonitis | 59 |
| NM-139276 | 0.0008 | −0.143 | 0.172 | Hs.463059 | 1 | Peritonitis | 60 |
| NOX2 | 0.0008 | −0.154 | 0.184 | Hs.292356 | 1 | Peritonitis | 61 |
| PLAT.2 | 0.0116 | −0.154 | 0.184 | Hs.491582 | 1 | Peritonitis | 62 |
| PPARD | 0.0000 | −0.179 | 0.215 | Hs.485196 | 1 | Peritonitis | 63 |
| R26118 | 0.0059 | 0.184 | −0.22 | Hs.594374 | 1 | Peritonitis | 64 |
| R36650 | 0.0052 | −0.126 | 0.151 | Hs.591522 | 1 | Peritonitis | 65 |
| R43074 | 0.0480 | 0.08 | −0.096 | Hs.298851 | 1 | Peritonitis | 66 |
| R53961 | 0.0118 | −0.106 | 0.127 | Hs.124128 | 1 | Peritonitis | 67 |
| R96155 | 0.0026 | 0.131 | −0.157 | | 1 | Peritonitis | 68 |
| TNFRSF6B.1 | 0.0045 | −0.122 | 0.146 | Hs.434878 | 1 | Peritonitis | 69 |
| TRIAD3 | 0.0039 | −0.143 | 0.172 | Hs.487458 | 1 | Peritonitis | 70 |
| TUCAN | 0.0001 | −0.153 | 0.183 | Hs.446146 | 1 | Peritonitis | 71 |
| W32272 | 0.0025 | 0.146 | −0.176 | Hs.594426 | 1 | Peritonitis | 72 |
| W85706 | 0.0000 | −0.214 | 0.257 | Hs.458973 | 1 | Peritonitis | 73 |
| XM-001687 | 0.0027 | −0.155 | 0.186 | Hs.77867 | 1 | Peritonitis | 74 |
| XM-006800 | 0.0000 | −0.235 | 0.282 | Hs.591043 | 1 | Peritonitis | 75 |
| XM-032902 | 0.0063 | 0.191 | −0.23 | Hs.593754 | 1 | Peritonitis | 76 |
| XM-036966 | 0.0190 | −0.097 | 0.116 | Hs.431850 | 1 | Peritonitis | 77 |

| Gen Bank | | | | | | | |
|---|---|---|---|---|---|---|---|
| Accession | | | | | | | |
| Nummer | p value | mean patient group 2 | mean patient group 1b | UniGene | Cluster | Subgroup | SeqID |
| AA017263 | 0.0099 | 0.16 | −0.127 | Hs.156727 | 2 | Pneumonia | 78 |
| AA017301 | 0.0179 | 0.139 | −0.11 | Hs.85863 | 2 | Pneumonia | 79 |
| AA031731 | 0.0015 | −0.358 | 0.283 | Hs.238964 | 2 | Pneumonia | 80 |
| AA398760 | 0.0275 | 0.213 | −0.168 | Hs.570638 | 2 | Pneumonia | 81 |
| AA400434 | 0.0088 | 0.137 | −0.108 | Hs.563200 | 2 | Pneumonia | 82 |
| AA400470 | 0.0418 | 0.146 | −0.115 | Hs.97805 | 2 | Pneumonia | 83 |
| AA402483 | 0.0138 | 0.169 | −0.134 | Hs.97313 | 2 | Pneumonia | 84 |
| AA417980 | 0.0004 | −0.281 | 0.223 | Hs.479226 | 2 | Pneumonia | 85 |
| AA428463 | 0.0195 | −0.186 | 0.147 | Hs.372739 | 2 | Pneumonia | 86 |
| AA436250 | 0.0019 | −0.361 | 0.286 | Hs.490203 | 2 | Pneumonia | 87 |
| AA452113 | 0.0054 | 0.144 | −0.114 | Hs.500643 | 2 | Pneumonia | 88 |
| AA458912 | 0.0330 | 0.167 | −0.132 | Hs.281898 | 2 | Pneumonia | 89 |
| AA459648 | 0.0086 | 0.154 | −0.122 | | 2 | Pneumonia | 90 |
| AA478611 | 0.0132 | −0.221 | 0.175 | Hs.105616 | 2 | Pneumonia | 91 |
| AA479727 | 0.0239 | 0.128 | −0.101 | Hs.23158 | 2 | Pneumonia | 92 |
| AA514450 | 0.0107 | 0.141 | −0.112 | | 2 | Pneumonia | 93 |
| AA541644 | 0.0000 | −0.218 | 0.173 | Hs.232165 | 2 | Pneumonia | 94 |
| AA548307 | 0.0234 | 0.137 | −0.108 | Hs.399800 | 2 | Pneumonia | 95 |
| AA626313 | 0.0137 | 0.142 | −0.113 | Hs.116150 | 2 | Pneumonia | 96 |
| AA628539 | 0.0280 | −0.132 | 0.105 | Hs.371001 | 2 | Pneumonia | 97 |
| AA699706 | 0.0043 | −0.268 | 0.212 | Hs.464779 | 2 | Pneumonia | 98 |
| AA844053 | 0.0402 | 0.119 | −0.094 | Hs.535257 | 2 | Pneumonia | 99 |
| AA887470 | 0.0423 | −0.12 | 0.095 | Hs.531081 | 2 | Pneumonia | 100 |
| AA897543 | 0.0122 | 0.235 | −0.186 | Hs.148217 | 2 | Pneumonia | 101 |
| AA906116 | 0.0083 | 0.264 | −0.209 | Hs.521545 | 2 | Pneumonia | 102 |
| AA910923 | 0.0030 | 0.194 | −0.153 | Hs.191164 | 2 | Pneumonia | 103 |
| AA935135 | 0.0222 | 0.148 | −0.117 | Hs.585129 | 2 | Pneumonia | 104 |
| AA969039 | 0.0051 | 0.152 | −0.121 | Hs.544636 | 2 | Pneumonia | 105 |
| AA992540 | 0.0002 | 0.258 | −0.204 | Hs.491869 | 2 | Pneumonia | 106 |
| ADRA2A | 0.0391 | −0.116 | 0.092 | Hs.249159 | 2 | Pneumonia | 107 |
| AF077011 | 0.0003 | 0.17 | −0.135 | Hs.459095 | 2 | Pneumonia | 108 |
| AI005466 | 0.0005 | 0.175 | −0.139 | Hs.602706 | 2 | Pneumonia | 109 |
| AI023336 | 0.0002 | 0.217 | −0.172 | Hs.370267 | 2 | Pneumonia | 110 |
| AI140065 | 0.0091 | −0.25 | 0.198 | Hs.146594 | 2 | Pneumonia | 111 |
| AI142427 | 0.0101 | −0.219 | 0.174 | Hs.300684 | 2 | Pneumonia | 112 |
| AI150305 | 0.0231 | 0.202 | −0.16 | Hs.128031 | 2 | Pneumonia | 113 |
| AI160757 | 0.0119 | 0.225 | −0.178 | Hs.408960 | 2 | Pneumonia | 114 |
| AI191762 | 0.0381 | 0.206 | −0.163 | Hs.495918 | 2 | Pneumonia | 115 |
| AI264774 | 0.0041 | 0.17 | −0.135 | Hs.514242 | 2 | Pneumonia | 116 |
| AI272798 | 0.0478 | −0.103 | 0.082 | Hs.479808 | 2 | Pneumonia | 117 |
| AI285411 | 0.0306 | −0.142 | 0.113 | Hs.635265 | 2 | Pneumonia | 118 |
| AI375046 | 0.0005 | 0.238 | −0.189 | | 2 | Pneumonia | 119 |
| AI421397 | 0.0391 | −0.117 | 0.093 | Hs.507025 | 2 | Pneumonia | 120 |
| AI492528 | 0.0268 | −0.158 | 0.125 | | 2 | Pneumonia | 121 |
| AI554942 | 0.0448 | 0.228 | −0.181 | Hs.570675 | 2 | Pneumonia | 122 |
| AI568793 | 0.0058 | −0.322 | 0.255 | Hs.368944 | 2 | Pneumonia | 123 |
| AI625724 | 0.0046 | −0.225 | 0.178 | Hs.185597 | 2 | Pneumonia | 124 |
| AI635650 | 0.0212 | 0.131 | −0.104 | | 2 | Pneumonia | 125 |
| AI654928 | 0.0000 | 0.272 | −0.216 | Hs.196133 | 2 | Pneumonia | 126 |
| AI685048 | 0.0004 | −0.165 | 0.131 | Hs.369785 | 2 | Pneumonia | 127 |
| AI700169 | 0.0010 | −0.334 | 0.264 | Hs.584910 | 2 | Pneumonia | 128 |
| AI745409 | 0.0032 | −0.265 | 0.21 | Hs.204924 | 2 | Pneumonia | 129 |
| AI798514 | 0.0314 | 0.143 | −0.113 | Hs.632218 | 2 | Pneumonia | 130 |
| AI799683 | 0.0047 | −0.149 | 0.118 | Hs.592083 | 2 | Pneumonia | 131 |
| AI799767 | 0.0323 | 0.22 | −0.175 | Hs.209226 | 2 | Pneumonia | 132 |
| AI801504 | 0.0108 | −0.135 | 0.107 | Hs.16064 | 2 | Pneumonia | 133 |
| AI808903 | 0.0240 | −0.15 | 0.118 | Hs.519855 | 2 | Pneumonia | 134 |
| AI811774 | 0.0115 | −0.148 | 0.117 | | 2 | Pneumonia | 135 |
| AI861979 | 0.0148 | 0.143 | −0.114 | Hs.469134 | 2 | Pneumonia | 136 |
| AI866656 | 0.0007 | 0.164 | −0.13 | Hs.211814 | 2 | Pneumonia | 137 |
| BC001604 | 0.0001 | −0.214 | 0.169 | Hs.182014 | 2 | Pneumonia | 138 |
| C2 | 0.0016 | 0.165 | −0.13 | Hs.408903 | 2 | Pneumonia | 139 |
| H02254 | 0.0280 | 0.245 | −0.194 | Hs.632489 | 2 | Pneumonia | 140 |
| H05436 | 0.0000 | 0.241 | −0.191 | Hs.11110 | 2 | Pneumonia | 141 |
| H11068 | 0.0045 | −0.174 | 0.138 | Hs.317243 | 2 | Pneumonia | 142 |
| H22946 | 0.0015 | 0.171 | −0.135 | Hs.534590 | 2 | Pneumonia | 143 |
| H38159 | 0.0026 | 0.211 | −0.167 | Hs.162996 | 2 | Pneumonia | 144 |
| H50201 | 0.0430 | −0.17 | 0.135 | Hs.21413 | 2 | Pneumonia | 145 |
| H73724 | 0.0002 | 0.211 | −0.167 | Hs.119882 | 2 | Pneumonia | 146 |
| H83996 | 0.0020 | 0.147 | −0.116 | Hs.153458 | 2 | Pneumonia | 147 |
| ICAM4 | 0.0113 | 0.118 | −0.093 | Hs.631609 | 2 | Pneumonia | 148 |
| IL18mRNA | 0.0001 | −0.182 | 0.144 | Hs.83077 | 2 | Pneumonia | 149 |
| IL26mRNA | 0.0293 | 0.108 | −0.085 | Hs.272350 | 2 | Pneumonia | 150 |
| LTBP4 | 0.0499 | −0.161 | 0.128 | Hs.466766 | 2 | Pneumonia | 151 |
| LTBR | 0.0191 | −0.164 | 0.13 | Hs.1116 | 2 | Pneumonia | 152 |
| M37435 | 0.0488 | −0.109 | 0.087 | Hs.173894 | 2 | Pneumonia | 153 |
| MAP4K4 | 0.0192 | −0.13 | 0.103 | Hs.431550 | 2 | Pneumonia | 154 |
| MAPK11.1 | 0.0268 | 0.116 | −0.092 | Hs.57732 | 2 | Pneumonia | 155 |
| MAPK7 | 0.0195 | −0.233 | 0.184 | Hs.150136 | 2 | Pneumonia | 156 |
| N29761 | 0.0156 | 0.552 | −0.437 | Hs.9315 | 2 | Pneumonia | 157 |
| N49848 | 0.0384 | 0.213 | −0.168 | Hs.104091 | 2 | Pneumonia | 158 |
| N55249 | 0.0426 | −0.185 | 0.147 | Hs.143347 | 2 | Pneumonia | 159 |
| N64649 | 0.0181 | 0.166 | −0.131 | Hs.102402 | 2 | Pneumonia | 160 |
| N69363 | 0.0284 | 0.158 | −0.125 | Hs.594444 | 2 | Pneumonia | 161 |
| N70546 | 0.0071 | −0.253 | 0.2 | Hs.155040 | 2 | Pneumonia | 162 |
| N80868 | 0.0128 | 0.143 | −0.113 | Hs.211426 | 2 | Pneumonia | 163 |
| NFKBIL2 | 0.0003 | −0.184 | 0.146 | Hs.459376 | 2 | Pneumonia | 164 |
| NM-001295 | 0.0001 | −0.189 | 0.149 | Hs.301921 | 2 | Pneumonia | 165 |
| NM-057158 | 0.0214 | 0.169 | −0.134 | Hs.417962 | 2 | Pneumonia | 166 |
| NOX1.2 | 0.0265 | 0.239 | −0.189 | Hs.592227 | 2 | Pneumonia | 167 |
| R00206 | 0.0028 | 0.199 | −0.158 | Hs.533551 | 2 | Pneumonia | 168 |
| R09463 | 0.0145 | 0.176 | −0.139 | Hs.344165 | 2 | Pneumonia | 169 |
| R16722 | 0.0334 | 0.221 | −0.175 | Hs.124246 | 2 | Pneumonia | 170 |
| R41771 | 0.0013 | 0.153 | −0.121 | Hs.591601 | 2 | Pneumonia | 171 |
| R42593 | 0.0207 | 0.154 | −0.122 | Hs.80395 | 2 | Pneumonia | 172 |
| R42778 | 0.0077 | 0.215 | −0.17 | Hs.553877 | 2 | Pneumonia | 173 |
| R43910 | 0.0009 | 0.398 | −0.315 | Hs.586760 | 2 | Pneumonia | 174 |
| R46801 | 0.0070 | −0.172 | 0.136 | Hs.343664 | 2 | Pneumonia | 175 |
| R49244 | 0.0078 | 0.125 | −0.099 | Hs.443258 | 2 | Pneumonia | 176 |
| R50755 | 0.0000 | 0.192 | −0.152 | Hs.633191 | 2 | Pneumonia | 177 |
| R60898 | 0.0032 | 0.136 | −0.108 | Hs.568242 | 2 | Pneumonia | 178 |
| R62926 | 0.0257 | 0.169 | −0.134 | Hs.285193 | 2 | Pneumonia | 179 |
| R79239 | 0.0087 | 0.18 | −0.143 | Hs.530588 | 2 | Pneumonia | 180 |
| R80259 | 0.0020 | 0.16 | −0.126 | Hs.595477 | 2 | Pneumonia | 181 |
| TGFB1 | 0.0136 | −0.122 | 0.097 | Hs.155218 | 2 | Pneumonia | 182 |
| W88496 | 0.0318 | 0.214 | −0.17 | Hs.314413 | 2 | Pneumonia | 183 |
| X57817 | 0.0119 | −0.639 | 0.506 | Hs.561078 | 2 | Pneumonia | 184 |
| XM-006953 | 0.0341 | 0.208 | −0.165 | Hs.355307 | 2 | Pneumonia | 185 |
| XM-028642 | 0.0149 | −0.175 | 0.139 | Hs.505654 | 2 | Pneumonia | 186 |
| XM-033972 | 0.0496 | −0.121 | 0.096 | Hs.492740 | 2 | Pneumonia | 187 |
| XM-034166 | 0.0028 | 0.149 | −0.118 | Hs.462525 | 2 | Pneumonia | 188 |
| XM-041844 | 0.0194 | 0.159 | −0.126 | Hs.36 | 2 | Pneumonia | 189 |
| XM-049849 | 0.0006 | 0.219 | −0.174 | Hs.512898 | 2 | Pneumonia | 190 |
| XM-057131 | 0.0000 | −0.313 | 0.248 | Hs.525157 | 2 | Pneumonia | 191 |

TABLE 4
Common set of sequences with similar significant gene activity in patients with severe infections which are
specific for a local inflammation, but not for peritonitis or pneumonia of a “fever of unknown origin”.

| GenBank | | | normalized and transformed expression signals | | | | | |
|---|---|---|---|---|---|---|---|---|
| Accession | p value | p value | mean patient | mean patient | mean patient | | | |
| Nummer | (patient group 1a) | (patient group 1b) | group 2 | group 1a | group 1b | UniGene | Cluster | SeqID |
| AA035428 | 0.006 | 0.007 | 0.15 | −0.17 | −0.15 | Hs.437006 | 3 | 192 |
| AA044390 | 0.015 | 0 | −0.22 | 0.27 | 0.27 | Hs.516217 | 3 | 193 |
| AA046302 | 0.032 | 0 | 0.15 | −0.17 | −0.35 | Hs.21611 | 3 | 194 |
| AA059314 | 0.037 | 0.011 | 0.15 | −0.18 | −0.22 | Hs.433445 | 3 | 195 |
| AA151104 | 0.001 | 0 | 0.24 | −0.29 | −0.29 | Hs.73454 | 3 | 196 |
| AA397913 | 0.006 | 0.04 | −0.12 | 0.14 | 0.11 | Hs.549040 | 3 | 197 |
| AA398331 | 0.018 | 0.007 | 0.16 | −0.19 | −0.2 | Hs.599407 | 3 | 198 |
| AA400790 | 0.011 | 0.006 | 0.22 | −0.26 | −0.24 | Hs.127999 | 3 | 199 |
| AA418841 | 0.039 | 0.03 | −0.16 | 0.19 | 0.13 | Hs.475319 | 3 | 200 |
| AA425808 | 0 | 0.005 | 0.22 | −0.27 | −0.14 | | 3 | 201 |
| AA426618 | 0.008 | 0.007 | 0.16 | −0.19 | −0.15 | Hs.585398 | 3 | 202 |
| AA455638 | 0.045 | 0.007 | −0.14 | 0.17 | 0.19 | Hs.444332 | 3 | 203 |
| AA461499 | 0.029 | 0.014 | 0.14 | −0.16 | −0.15 | Hs.99546 | 3 | 204 |
| AA478985 | 0.012 | 0.008 | 0.17 | −0.2 | −0.15 | Hs.279784 | 3 | 205 |
| AA514237 | 0 | 0.001 | −0.23 | 0.28 | 0.19 | Hs.147880 | 3 | 206 |
| AA620760 | 0.007 | 0.016 | 0.21 | −0.25 | −0.21 | Hs.633241 | 3 | 207 |
| AA682790 | 0.002 | 0.04 | 0.16 | −0.19 | −0.11 | | 3 | 208 |
| AA702492 | 0 | 0.002 | −0.29 | 0.35 | 0.17 | Hs.368563 | 3 | 209 |
| AA703200 | 0.01 | 0.001 | 0.17 | −0.2 | −0.18 | Hs.597767 | 3 | 210 |
| AA740907 | 0.004 | 0 | 0.15 | −0.18 | −0.27 | Hs.88297 | 3 | 211 |
| AA759092 | 0.016 | 0.025 | 0.11 | −0.14 | −0.15 | Hs.121439 | 3 | 212 |
| AA781411 | 0.005 | 0.021 | 0.16 | −0.2 | −0.12 | Hs.166015 | 3 | 213 |
| AA807376 | 0.009 | 0.015 | 0.14 | −0.17 | −0.17 | Hs.370414 | 3 | 214 |
| AA813145 | 0.018 | 0 | 0.16 | −0.19 | −0.38 | Hs.601872 | 3 | 215 |
| AA845015 | 0.041 | 0.02 | 0.12 | −0.15 | −0.13 | Hs.631866 | 3 | 216 |
| AA845475 | 0 | 0.022 | −0.2 | 0.24 | 0.1 | Hs.487393 | 3 | 217 |
| AA860398 | 0.014 | 0.018 | 0.12 | −0.14 | −0.15 | Hs.569748 | 3 | 218 |
| AA894523 | 0.013 | 0 | 0.13 | −0.15 | −0.18 | Hs.10734 | 3 | 219 |
| AA947111 | 0.004 | 0.006 | 0.13 | −0.15 | −0.11 | Hs.562136 | 3 | 220 |
| ADRB1 | 0 | 0 | −0.2 | 0.24 | 0.25 | Mm.46797 | 3 | 221 |
| AHSG | 0.022 | 0.02 | 0.22 | −0.26 | −0.18 | Hs.324746 | 3 | 222 |
| AI023558 | 0.001 | 0.001 | 0.15 | −0.18 | −0.17 | Hs.131417 | 3 | 223 |
| AI041544 | 0 | 0 | −0.22 | 0.26 | 0.16 | Hs.575480 | 3 | 224 |
| AI091867 | 0 | 0 | −0.22 | 0.26 | 0.24 | Hs.631761 | 3 | 225 |
| AI093704 | 0.009 | 0.012 | −0.18 | 0.21 | 0.19 | Hs.603146 | 3 | 226 |
| AI097494 | 0.002 | 0.005 | 0.17 | −0.21 | −0.16 | Hs.307984 | 3 | 227 |
| AI148246 | 0.024 | 0.004 | 0.14 | −0.17 | −0.18 | Hs.474251 | 3 | 228 |
| AI167874 | 0.006 | 0.002 | 0.13 | −0.15 | −0.16 | Hs.71023 | 3 | 229 |
| AI203091 | 0.016 | 0.027 | 0.13 | −0.15 | −0.12 | Hs.604090 | 3 | 230 |
| AI203697 | 0.001 | 0.016 | −0.15 | 0.18 | 0.09 | Hs.567342 | 3 | 231 |
| AI220662 | 0.049 | 0.019 | −0.12 | 0.15 | 0.1 | Hs.508720 | 3 | 232 |
| AI222359 | 0.005 | 0.003 | 0.23 | −0.28 | −0.29 | Hs.556230 | 3 | 233 |
| AI223092 | 0.004 | 0.015 | 0.16 | −0.19 | −0.18 | Hs.147880 | 3 | 234 |
| AI264626 | 0.001 | 0.005 | 0.16 | −0.19 | −0.12 | | 3 | 235 |
| AI267659 | 0.026 | 0.031 | 0.13 | −0.15 | −0.11 | | 3 | 236 |
| AI271764 | 0 | 0.003 | −0.31 | 0.37 | 0.28 | Hs.446357 | 3 | 237 |
| AI342905 | 0.032 | 0.04 | 0.15 | −0.18 | −0.17 | Hs.604613 | 3 | 238 |
| AI357099 | 0.005 | 0.025 | 0.28 | −0.34 | −0.26 | Hs.584910 | 3 | 239 |
| AI373295 | 0.012 | 0.034 | 0.15 | −0.18 | −0.13 | Hs.544825 | 3 | 240 |
| AI373525 | 0.018 | 0.001 | 0.11 | −0.13 | −0.14 | Hs.539391 | 3 | 241 |
| AI378275 | 0.006 | 0.018 | 0.18 | −0.21 | −0.15 | Hs.61271 | 3 | 242 |
| AI453476 | 0.02 | 0.031 | 0.15 | −0.18 | −0.14 | Hs.168677 | 3 | 243 |
| AI478776 | 0.001 | 0.042 | −0.18 | 0.22 | 0.15 | | 3 | 244 |
| AI539271 | 0.027 | 0.016 | 0.19 | −0.23 | −0.23 | Hs.478000 | 3 | 245 |
| AI554283 | 0.043 | 0.021 | 0.14 | −0.17 | −0.16 | Hs.420529 | 3 | 246 |
| AI565469 | 0.015 | 0 | 0.16 | −0.19 | −0.22 | Hs.638685 | 3 | 247 |
| AI582909 | 0 | 0 | 0.2 | −0.24 | −0.22 | Hs.578450 | 3 | 248 |
| AI589096 | 0.002 | 0.036 | −0.23 | 0.27 | 0.18 | Hs.638946 | 3 | 249 |
| AI590144 | 0.003 | 0.022 | 0.27 | −0.33 | −0.24 | Hs.508848 | 3 | 250 |
| AI613038 | 0.012 | 0 | −0.15 | 0.17 | 0.26 | | 3 | 251 |
| AI627286 | 0.008 | 0.003 | 0.12 | −0.14 | −0.14 | Hs.191073 | 3 | 252 |
| AI628322 | 0.006 | 0.038 | 0.22 | −0.27 | −0.12 | Hs.530538 | 3 | 253 |
| AI652609 | 0.027 | 0.012 | 0.12 | −0.14 | −0.12 | Hs.567425 | 3 | 254 |
| AI692869 | 0.002 | 0.028 | 0.24 | −0.28 | −0.19 | Hs.202419 | 3 | 255 |
| AI696291 | 0.027 | 0.033 | 0.21 | −0.25 | −0.25 | Hs.528671 | 3 | 256 |
| AI700444 | 0.029 | 0.019 | 0.18 | −0.21 | −0.23 | Hs.564343 | 3 | 257 |
| AI733177 | 0 | 0.004 | −0.22 | 0.27 | 0.22 | Hs.421340 | 3 | 258 |
| AI733269 | 0 | 0.003 | −0.26 | 0.31 | 0.18 | Hs.418045 | 3 | 259 |
| AI733498 | 0.045 | 0.001 | 0.16 | −0.19 | −0.32 | Hs.573606 | 3 | 260 |
| AI738831 | 0 | 0.032 | 0.26 | −0.32 | −0.14 | Hs.268606 | 3 | 261 |
| AI739381 | 0.005 | 0.004 | 0.14 | −0.16 | −0.2 | Hs.512763 | 3 | 262 |
| AI742529 | 0 | 0.003 | −0.22 | 0.27 | 0.16 | Hs.591095 | 3 | 263 |
| AI799137 | 0.048 | 0.006 | −0.16 | 0.19 | 0.27 | Hs.432690 | 3 | 264 |
| AI808410 | 0.006 | 0.008 | −0.16 | 0.19 | 0.15 | Hs.233955 | 3 | 265 |
| AI859370 | 0.027 | 0.015 | 0.12 | −0.14 | −0.14 | Hs.607181 | 3 | 266 |
| AI859777 | 0.013 | 0.011 | 0.22 | −0.26 | −0.25 | Hs.301819 | 3 | 267 |
| AI860121 | 0.001 | 0.004 | −0.21 | 0.25 | 0.23 | Hs.501684 | 3 | 268 |
| AI889310 | 0 | 0 | −0.21 | 0.25 | 0.17 | Hs.511903 | 3 | 269 |
| AI890962 | 0.026 | 0.017 | 0.13 | −0.16 | −0.12 | Hs.25601 | 3 | 270 |
| AI913322 | 0.009 | 0.023 | −0.24 | 0.29 | 0.19 | Hs.107740 | 3 | 271 |
| AI924028 | 0.003 | 0.003 | 0.28 | −0.34 | −0.27 | Hs.326391 | 3 | 272 |
| AI924296 | 0.017 | 0.041 | 0.11 | −0.13 | −0.11 | Hs.487479 | 3 | 273 |
| AI925451 | 0.046 | 0.008 | 0.16 | −0.19 | −0.18 | Hs.629605 | 3 | 274 |
| AI926659 | 0.001 | 0.04 | −0.23 | 0.27 | 0.15 | Hs.363558 | 3 | 275 |
| AI933607 | 0 | 0.002 | −0.25 | 0.31 | 0.17 | Hs.632296 | 3 | 276 |
| AI936462 | 0.016 | 0.012 | −0.13 | 0.16 | 0.11 | | 3 | 277 |
| ARHA | 0.001 | 0.004 | −0.18 | 0.22 | 0.13 | Hs.247077 | 3 | 278 |
| ASC | 0.001 | 0.014 | −0.2 | 0.24 | 0.11 | Hs.499094 | 3 | 279 |
| B3GALT3 | 0.005 | 0.01 | 0.21 | −0.25 | −0.18 | Hs.418062 | 3 | 280 |
| BC013302 | 0.003 | 0.033 | −0.13 | 0.16 | 0.08 | Hs.437594 | 3 | 281 |
| BC021289 | 0.023 | 0.005 | −0.27 | 0.33 | 0.27 | Hs.75431 | 3 | 282 |
| BC024270 | 0 | 0.017 | −0.15 | 0.18 | 0.13 | Hs.136164 | 3 | 283 |
| BCL6 | 0.003 | 0.011 | −0.26 | 0.32 | 0.21 | Hs.636990 | 3 | 284 |
| BZRP | 0 | 0 | −0.47 | 0.56 | 0.37 | Hs.202 | 3 | 285 |
| BZRP.1 | 0.002 | 0 | −0.34 | 0.41 | 0.35 | Hs.202 | 3 | 286 |
| CIQBP | 0 | 0.001 | −0.24 | 0.28 | 0.23 | Hs.622699 | 3 | 287 |
| CIS | 0.015 | 0.037 | 0.12 | −0.15 | −0.09 | Hs.458355 | 3 | 288 |
| C8B | 0.005 | 0 | 0.13 | −0.16 | −0.2 | Hs.391835 | 3 | 289 |
| C9 | 0 | 0.001 | −0.25 | 0.3 | 0.15 | Hs.481980 | 3 | 290 |
| CCR7 | 0.037 | 0.024 | 0.11 | −0.13 | −0.12 | Hs.370036 | 3 | 291 |
| CD59 | 0.007 | 0.015 | −0.2 | 0.24 | 0.17 | Hs.633297 | 3 | 292 |
| CDKN1A | 0.049 | 0.007 | −0.12 | 0.14 | 0.18 | Hs.370771 | 3 | 293 |
| CKM | 0.004 | 0.003 | 0.2 | −0.24 | −0.22 | Hs.334347 | 3 | 294 |
| EDARADD | 0 | 0 | −0.24 | 0.29 | 0.2 | Hs.352224 | 3 | 295 |
| EPB41 | 0.018 | 0.013 | 0.2 | −0.25 | −0.25 | Hs.175437 | 3 | 296 |
| FADD | 0 | 0.009 | −0.21 | 0.25 | 0.12 | Hs.86131 | 3 | 297 |
| FLOT2 | 0 | 0 | −0.35 | 0.42 | 0.29 | Hs.514038 | 3 | 298 |
| G3BP2 | 0.001 | 0.003 | −0.19 | 0.23 | 0.22 | Hs.593867 | 3 | 299 |
| GADD45A | 0.001 | 0.007 | −0.27 | 0.33 | 0.21 | Hs.80409 | 3 | 300 |
| H16716 | 0.002 | 0 | −0.25 | 0.31 | 0.25 | Hs.448889 | 3 | 301 |
| H18435 | 0.012 | 0.001 | 0.18 | −0.22 | −0.31 | | 3 | 302 |
| H18649 | 0 | 0.007 | 0.29 | −0.34 | −0.19 | | 3 | 303 |
| H23819 | 0.015 | 0.004 | 0.17 | −0.2 | −0.22 | Hs.59093 | 3 | 304 |
| H30516 | 0.012 | 0.031 | 0.17 | −0.2 | −0.14 | Hs.482411 | 3 | 305 |
| H44908 | 0 | 0 | −0.28 | 0.34 | 0.23 | Hs.591171 | 3 | 306 |
| H61449 | 0.009 | 0 | 0.13 | −0.15 | −0.18 | Hs.528368 | 3 | 307 |
| H98244 | 0.001 | 0.019 | 0.23 | −0.28 | −0.17 | Hs.143707 | 3 | 308 |
| H99099 | 0.003 | 0.029 | −0.22 | 0.26 | 0.17 | Hs.205742 | 3 | 309 |
| HSPA1A | 0.011 | 0.019 | −0.16 | 0.19 | 0.13 | Hs.520028 | 3 | 310 |
| HSPA8.1 | 0.005 | 0.006 | −0.2 | 0.23 | 0.23 | Hs.180414 | 3 | 311 |
| HSPB1 | 0 | 0.013 | −0.41 | 0.5 | 0.14 | Hs.520973 | 3 | 312 |
| ICAM5 | 0.003 | 0.003 | −0.13 | 0.15 | 0.13 | Hs.465862 | 3 | 313 |
| IFNA5 | 0.012 | 0 | −0.16 | 0.19 | 0.21 | Hs.37113 | 3 | 314 |
| IKBKG | 0.001 | 0 | −0.13 | 0.16 | 0.14 | Hs.43505 | 3 | 315 |
| IL10RB | 0 | 0.004 | −0.23 | 0.28 | 0.17 | Hs.512211 | 3 | 316 |
| IL12B | 0.036 | 0.015 | 0.32 | −0.39 | −0.28 | Hs.674 | 3 | 317 |
| IL1RN | 0.002 | 0 | −0.31 | 0.37 | 0.27 | Hs.594611 | 3 | 318 |
| IL2RG | 0 | 0.013 | −0.24 | 0.29 | 0.12 | Hs.84 | 3 | 319 |
| IL6R | 0.002 | 0.001 | −0.16 | 0.19 | 0.19 | Hs.591492 | 3 | 320 |
| IL7R | 0.019 | 0.04 | 0.21 | −0.26 | −0.17 | Hs.591742 | 3 | 321 |
| M37435 | 0.013 | 0.017 | −0.13 | 0.15 | 0.13 | Hs.173894 | 3 | 322 |
| M61199 | 0.003 | 0.028 | −0.22 | 0.26 | 0.16 | Hs.591602 | 3 | 323 |
| MAP3K11 | 0.024 | 0.01 | −0.14 | 0.16 | 0.13 | Hs.502872 | 3 | 324 |
| MAP3K6 | 0.032 | 0.041 | −0.12 | 0.15 | 0.11 | Hs.194694 | 3 | 325 |
| MAPK14 | 0 | 0.004 | −0.24 | 0.28 | 0.19 | Hs.588289 | 3 | 326 |
| MAPK14.3 | 0 | 0.001 | −0.26 | 0.31 | 0.25 | Hs.588289 | 3 | 327 |
| MAPK8.3 | 0.025 | 0.016 | 0.11 | −0.13 | −0.14 | Hs.138211 | 3 | 328 |
| MYD88 | 0 | 0 | −0.25 | 0.3 | 0.25 | Hs.82116 | 3 | 329 |
| MYL2 | 0.032 | 0 | −0.17 | 0.2 | 0.23 | Hs.491359 | 3 | 330 |
| N32857 | 0.004 | 0.02 | −0.54 | 0.65 | 0.45 | Hs.491767 | 3 | 331 |
| N51537 | 0.042 | 0.002 | 0.15 | −0.18 | −0.19 | Hs.525485 | 3 | 332 |
| N52915 | 0.005 | 0 | 0.16 | −0.19 | −0.18 | | 3 | 333 |
| N53973 | 0.014 | 0.006 | 0.13 | −0.16 | −0.15 | Hs.585782 | 3 | 334 |
| N67859 | 0.004 | 0.041 | −0.16 | 0.19 | 0.12 | Hs.125829 | 3 | 335 |
| NET1 | 0.021 | 0.013 | −0.11 | 0.13 | 0.13 | Hs.610771 | 3 | 336 |
| NM_001540 | 0 | 0.013 | −0.2 | 0.24 | 0.13 | Hs.626419 | 3 | 337 |
| NM_003258 | 0.005 | 0 | −0.21 | 0.25 | 0.27 | Hs.515122 | 3 | 338 |
| NM_006936 | 0.001 | 0.001 | −0.25 | 0.3 | 0.2 | Hs.474005 | 3 | 339 |
| NM_016184 | 0.017 | 0.022 | 0.22 | −0.27 | −0.24 | Hs.504657 | 3 | 340 |
| NM_025139 | 0.014 | 0.003 | 0.19 | −0.23 | −0.29 | Hs.471610 | 3 | 341 |
| NM_031311 | 0 | 0.024 | 0.5 | −0.6 | −0.24 | Hs.233389 | 3 | 342 |
| NM-000061 | 0.005 | 0.047 | −0.15 | 0.18 | 0.1 | Hs.159494 | 3 | 343 |
| NM-000584 | 0.017 | 0.007 | −0.18 | 0.21 | 0.21 | Hs.632880 | 3 | 344 |
| NM-000760 | 0.002 | 0.011 | −0.18 | 0.21 | 0.13 | Hs.524517 | 3 | 345 |
| NM-001013 | 0.015 | 0.027 | −0.11 | 0.13 | 0.11 | Hs.546288 | 3 | 346 |
| NM-001101 | 0 | 0 | −0.27 | 0.33 | 0.19 | Hs.593869 | 3 | 347 |
| NM-001288 | 0.007 | 0.001 | −0.14 | 0.17 | 0.18 | Mm.388801 | 3 | 348 |
| NM-001315 | 0 | 0.001 | −0.25 | 0.3 | 0.22 | Hs.588289 | 3 | 349 |
| NM-001569 | 0.001 | 0.012 | −0.16 | 0.19 | 0.11 | Hs.522819 | 3 | 350 |
| NM-001765 | 0.041 | 0.005 | −0.12 | 0.14 | 0.16 | Hs.1311 | 3 | 351 |
| NM-001772 | 0.018 | 0.004 | −0.12 | 0.14 | 0.12 | Hs.83731 | 3 | 352 |
| NM-002128 | 0.009 | 0.044 | −0.2 | 0.24 | 0.15 | Hs.593339 | 3 | 353 |
| NM-002394 | 0 | 0.012 | −0.19 | 0.23 | 0.12 | Hs.502769 | 3 | 354 |
| NM-002401 | 0.037 | 0.003 | −0.14 | 0.17 | 0.17 | Hs.29282 | 3 | 355 |
| NM-002415 | 0 | 0.025 | −0.24 | 0.28 | 0.14 | Hs.632781 | 3 | 356 |
| NM-002953 | 0 | 0 | −0.19 | 0.23 | 0.21 | Hs.149957 | 3 | 357 |
| NM-003153 | 0 | 0.041 | −0.2 | 0.24 | 0.13 | Hs.524518 | 3 | 358 |
| NM-003268 | 0 | 0.005 | −0.31 | 0.38 | 0.45 | Hs.135853 | 3 | 359 |
| NM-003684 | 0.017 | 0.001 | −0.17 | 0.2 | 0.16 | Hs.371594 | 3 | 360 |
| NM-004257 | 0 | 0 | −0.25 | 0.3 | 0.19 | Hs.446350 | 3 | 361 |
| NM-004635 | 0.02 | 0.031 | −0.1 | 0.12 | 0.11 | Hs.624942 | 3 | 362 |
| NM-004740 | 0 | 0.001 | −0.25 | 0.3 | 0.19 | Hs.462590 | 3 | 363 |
| NM-005620 | 0.001 | 0.001 | −0.23 | 0.27 | 0.22 | Hs.547382 | 3 | 364 |
| NM-005803 | 0.002 | 0.001 | −0.15 | 0.18 | 0.15 | Hs.179986 | 3 | 365 |
| NM-007328 | 0.005 | 0 | 0.36 | −0.43 | −0.38 | Hs.512576 | 3 | 366 |
| NM-018643 | 0 | 0.035 | 0.39 | −0.47 | −0.14 | Hs.283022 | 3 | 367 |
| NM-138556 | 0.03 | 0.009 | 0.17 | −0.2 | −0.1 | Hs.174312 | 3 | 368 |
| PPGB | 0 | 0.015 | −0.22 | 0.26 | 0.14 | Hs.609336 | 3 | 369 |
| PPP4C | 0 | 0 | −0.19 | 0.23 | 0.15 | Hs.534338 | 3 | 370 |
| PRP4.1 | 0.035 | 0.005 | −0.2 | 0.24 | 0.24 | Mm.10027 | 3 | 371 |
| R00742 | 0.007 | 0.037 | 0.16 | −0.19 | −0.15 | Hs.275675 | 3 | 372 |
| R05804 | 0.034 | 0.02 | 0.12 | −0.15 | −0.14 | Hs.348308 | 3 | 373 |
| R26635 | 0 | 0.041 | −0.49 | 0.59 | 0.16 | Hs.178499 | 3 | 374 |
| R37251 | 0.033 | 0.038 | −0.16 | 0.19 | 0.17 | Hs.479099 | 3 | 375 |
| R39782 | 0 | 0.002 | 0.22 | −0.26 | −0.2 | Hs.21145 | 3 | 376 |
| R40406 | 0.022 | 0.035 | −0.14 | 0.16 | 0.2 | Hs.2853 | 3 | 377 |
| R42461 | 0.004 | 0.008 | −0.2 | 0.23 | 0.26 | Hs.579115 | 3 | 378 |
| R42782 | 0.026 | 0.017 | −0.17 | 0.21 | 0.14 | Hs.434971 | 3 | 379 |
| R43203 | 0.011 | 0.027 | 0.3 | −0.36 | −0.25 | Hs.73828 | 3 | 380 |
| R43722 | 0.033 | 0.032 | −0.2 | 0.23 | 0.21 | | 3 | 381 |
| R45159 | 0 | 0 | 0.23 | −0.28 | −0.22 | Hs.271285 | 3 | 382 |
| R45355 | 0 | 0 | −0.18 | 0.21 | 0.2 | Hs.201805 | 3 | 383 |
| R52949 | 0.024 | 0.035 | 0.16 | −0.19 | −0.17 | Hs.622398 | 3 | 384 |
| R54442 | 0.001 | 0.006 | 0.24 | −0.28 | −0.23 | Hs.416139 | 3 | 385 |
| R56890 | 0.011 | 0.002 | 0.14 | −0.16 | −0.14 | Hs.592205 | 3 | 386 |
| R58974 | 0.033 | 0.034 | 0.13 | −0.16 | −0.2 | Hs.335205 | 3 | 387 |
| R84393 | 0.027 | 0.023 | 0.15 | −0.17 | −0.11 | Hs.387255 | 3 | 388 |
| R89802 | 0.006 | 0 | 0.18 | −0.22 | −0.17 | Hs.93670 | 3 | 389 |
| RAC1 | 0.01 | 0.01 | −0.11 | 0.13 | 0.14 | Hs.413812 | 3 | 390 |
| RAC3 | 0 | 0 | −0.35 | 0.41 | 0.26 | Hs.45002 | 3 | 391 |
| SERCA2-2 | 0 | 0.004 | −0.17 | 0.2 | 0.15 | Hs.633656 | 3 | 392 |
| SPTLC2 | 0 | 0.007 | −0.19 | 0.23 | 0.2 | Hs.435661 | 3 | 393 |
| T65410 | 0.018 | 0.042 | −0.2 | 0.24 | 0.19 | Hs.125116 | 3 | 394 |
| T90460 | 0.005 | 0.023 | −0.15 | 0.18 | 0.12 | Hs.102471 | 3 | 395 |
| T91086 | 0.044 | 0.015 | 0.2 | −0.24 | −0.21 | | 3 | 396 |
| T91795 | 0.038 | 0.041 | 0.14 | −0.17 | −0.16 | Hs.502872 | 3 | 397 |
| T95815 | 0.038 | 0.032 | 0.09 | −0.11 | −0.12 | Hs.336994 | 3 | 398 |
| TNFSF14 | 0.004 | 0.001 | 0.13 | −0.16 | −0.17 | Hs.129708 | 3 | 399 |
| TNFSF9 | 0.001 | 0.03 | −0.19 | 0.22 | 0.14 | Hs.1524 | 3 | 400 |
| TRAF3 | 0.014 | 0.041 | −0.15 | 0.18 | 0.11 | Hs.510528 | 3 | 401 |
| U07802 | 0.004 | 0.023 | 0.24 | −0.28 | −0.26 | Hs.503093 | 3 | 402 |
| UGCG | 0.009 | 0 | −0.26 | 0.31 | 0.53 | Hs.304249 | 3 | 403 |
| W86767 | 0.046 | 0.001 | 0.1 | −0.12 | −0.17 | Hs.632594 | 3 | 404 |
| X05875 | 0.011 | 0.021 | −0.37 | 0.45 | 0.23 | Hs.99863 | 3 | 405 |
| X64641 | 0.004 | 0.015 | −0.46 | 0.56 | 0.44 | Hs.449621 | 3 | 406 |
| XM-003937 | 0.002 | 0.001 | 0.26 | −0.32 | −0.38 | Hs.591258 | 3 | 407 |
| XM-004256 | 0.014 | 0 | −0.31 | 0.38 | 0.6 | Hs.606320 | 3 | 408 |
| XM-006867 | 0.002 | 0.001 | −0.19 | 0.22 | 0.19 | Hs.591014 | 3 | 409 |
| XM-007417 | 0.031 | 0.016 | 0.1 | −0.12 | −0.27 | Hs.636674 | 3 | 410 |
| XM-008679 | 0.009 | 0.019 | −0.28 | 0.33 | 0.24 | Hs.408312 | 3 | 411 |
| XM-009475 | 0.001 | 0.001 | −0.36 | 0.44 | 0.4 | Hs.388004 | 3 | 412 |
| XM-012039 | 0 | 0.004 | −0.28 | 0.33 | 0.24 | Hs.568921 | 3 | 413 |
| XM-015278 | 0 | 0.001 | −0.19 | 0.23 | 0.21 | Hs.334019 | 3 | 414 |
| XM-015396 | 0 | 0 | −0.41 | 0.49 | 0.35 | Hs.507658 | 3 | 415 |
| XM-015815 | 0.008 | 0.007 | −0.23 | 0.27 | 0.25 | Hs.509067 | 3 | 416 |
| XM-027358 | 0 | 0.042 | −0.29 | 0.35 | 0.16 | Hs.592992 | 3 | 417 |
| XM-030326 | 0.002 | 0.021 | −0.22 | 0.26 | 0.17 | Hs.626357 | 3 | 418 |
| XM-030906 | 0 | 0 | −0.23 | 0.27 | 0.22 | Hs.155218 | 3 | 419 |
| XM-031242 | 0 | 0 | −0.25 | 0.3 | 0.29 | Hs.527778 | 3 | 420 |
| XM-033862 | 0.004 | 0.001 | −0.13 | 0.15 | 0.14 | Hs.181128 | 3 | 421 |
| XM-038024 | 0.024 | 0.033 | 0.14 | −0.17 | −0.11 | Hs.318547 | 3 | 422 |
| XM-041101 | 0.004 | 0 | −0.19 | 0.22 | 0.18 | Hs.504877 | 3 | 423 |
| XM-042066 | 0.006 | 0.025 | −0.14 | 0.17 | 0.15 | Hs.508461 | 3 | 424 |
| XM-046575 | 0.028 | 0.007 | 0.18 | −0.21 | −0.2 | Hs.562457 | 3 | 425 |
| XM-047570 | 0 | 0 | −0.26 | 0.32 | 0.21 | Hs.495912 | 3 | 426 |
| XM-048068 | 0.001 | 0 | −0.24 | 0.29 | 0.23 | Hs.531668 | 3 | 427 |
| XM-052636 | 0 | 0 | −0.27 | 0.32 | 0.23 | Hs.634911 | 3 | 428 |
| XM-055188 | 0.029 | 0.011 | −0.17 | 0.2 | 0.18 | Hs.76753 | 3 | 429 |
| XM-056556 | 0 | 0.019 | −0.34 | 0.41 | 0.17 | Hs.501497 | 3 | 430 |
| XM-057356 | 0 | 0.009 | −0.22 | 0.26 | 0.23 | Hs.268675 | 3 | 431 |
| XM-114018 | 0.001 | 0.012 | −0.17 | 0.21 | 0.12 | Hs.591382 | 3 | 432 |

Thus, the specific gene activity cluster 1 and 2 ascertained are usable for the invention for establishing peritonitis or pneumonia as local inflammation for “fever of unknown origin”.

The gene activity cluster 3 is usable for the invention for establishing a local inflammation of a FUO which is not peritonitis or pneumonia.

### REFERENCES

1. Roth A. R., Basello. D. O., (2003), Approach to the adult patient with fever of unknown origin. Am. Fain. Phys., 68(11) 2223-2228.
2. Amin K., Kauffman C. A., (2003). Fever of unknown origin, postgrad. med., 114(3), 69-75.
3. Mourad, O., Palda, V., Detsky, (2003), A comprehensive evidence-based approach to fever of unknown origin, Arch. Intern. Med., 163, 545-551.
4. Liu, K. S., Shen, W. S., Chen, Y. C, Chang, S. C., Hsieh. (2003), Fever of unknown origin: a retrospective study of 78 adult patients in Taiwan, J Microbiol. Immunol. Infect., 36, 243-247.
5. Pile. J. C., (2006), Evaluating postoperative fever: a focused approach, Clev. Clin. J. Med., 73 (supp.1), S62-S66.
6. Sauer, H-J., (2001), Surveillance nosokomialer Infektionen auf Intensivstationen-Etablierung einer computergestüzten Inktionserfassung und-auswertung auf einer interdiziplinären 16-Betten-Intensivstation (surveillance of nosocomial infections on intensive care units—establishment of a computer-based recordation and analysis of infections on an interdisciplinary intensive care unit with 16 beds), MD thesis, Halle (Scale), Martin-Luther-University Halle-Wittenberg, Faculty of Medicine.
7. Vincent, J-L., Biharin D. J., Suter, P. M., Bruining, H. A., White J., Nicolas-Chanoin, M-H., Wolff, M., Spencer, R. C. Hemmer, M., (2000), the prevalence of nosocomial infection in intensive care units in Europe: Results of the European prevalence of infection in intensive care (EPIC) study, JAMA, 37, 454-460.
8. Welte, T., Marre, R., Suttorp, N., (2004), Das Kompetenznetzwerk “Ambulant erworbene Pneumonic” (The competence network “ambulantory-acquired pneumonia”) (CAPNETZ), Internist, 45, 393-401.
9. Unertl, K. Heiniger, A., Ventilator-associated Pneumonia, http://www.tu-dresden.de/medkai/unertl.pdf
10. Patel, P. J., Leeper Jr, K. V., McGowan Jr, J. E., (2002). Epidemiology and microbiology of hospital-acquired pneumonia, Semin. Respir. Crit. Care Med., 23(5), 415-425.
11. Park, D. R., The microbiology of ventilator-associated Pneumonia, Respir. Care, 50(86), 742-765.
12. Hall, J. C., Heel, K. A., Papadimitriou, J. M., Platell C., (1998), The pathology of peritonitis. Gastroenterology, 114, 185-196.
13. Troidle, L., Gorban-Brennan, N., Kliger, A., Finkelstein, F., (1998). Differing outcomes of Gram-positive and Gram-negative peritonitis, Am. J. Kidney Dis., 32(4), 623-628.
14. Jönsson, B., Berlund, J., Skau, T., Nyström, P. O., (1993), Outcome of intr-abdominal infection in pigs depends more on host responses than on microbiology, Eur. J. Surg., 159, 571-578.
15. Cobb, P. J., Laramie, J. M., Stormo, G. D., Morrissey, J. J., Shannon, W. D., Qiu, Y., Karl, 1., Buchman, T. G., Hotchkiss, R. S., (2002), Sepsis gene expression profiling: Murine splenic compared with hepatic responses determined using complementary microarrays, Crit Care Med., 30(12), 2711-2721.
16. Jonhson, S. B., (2006). Gene expression profiles differentiate between SIRS and early Sepsis, 126th Annual Meeting, American Surgical Assoc., Boston.
17. Mclean, A., (2006), Use of signature genes to diagnose sepsis in patients with SIRS, 26th International Symposium on Intensive Care and Emergency Medicine, Brussels.
18. Prucha M. Ruryk A, Boriss H. Moller E, Zazula R, Herold I, Claus R A, Reinhart K A, Deigner P, Russwurm S., (2004), Expression profiling: toward an application in sepsis diagnostics, Shock. 22(1):29-33.
19. Domashowske J. B. Bonville C. A., Easton, A. J., Rosenberg H. F., (2002), Differential expression of pro-inflammatory cytokine genes in vivo in response to pathogenic and non-pathogenic pneumovirus infections., J. Infect. Dis., 186(1), 8-14.
20. WO 2004/087949, Verfahren zur Erkennung akuter, generalisierter entzündlicher Zustände (SIRS), Sepsis, sepsisähnliehen Zustanden und systemischen Infektionen (Method for recognizing acute generalised inflammatory conditions (SIRS), sepsis, sepsis-like conditions and systemic infections)
21. WO 03/002763, Use of a biochip for the diagnosis of sepsis or sepsis related syndrome
22. DE 102004049897, Verfahren zur Unterscheidung zwischen nichtinfektiösen und infektiosen Ursachen eines Multiorganversagens (Method for differentiating between non-infectious and infectious causes of multiple organ failure)
23. Marshall, D. R., Olivas, E., Andreansky, S., La Gruta. N. L., Neale, G. A., Gutierrez, A., Wichlan, D. G., Cheng, C Doherty, P. C., Turner, S. J., (2005). Effector CD8+ T cells recovered from influenza pneumonia differentiate to state of focused gene expression, Proc. Natl. Acad. Sci. USA, 102(17), 6074-6079.
24. Selman. M., pardo, A., Barrera, L., Estradda, A., Watson, S. R., Wilson, K., Aziz, N., Kaminski, N., Zlotnik A., (2006), Gene expression profiles distinguish idiopathic pulmonary fibrosis from hypersensistivity pneumonitis, Am J. Respir. Crit. Care Med., 173(2), 188-198.
25. Sandler, N. G., Menttink-Kane, M. M., Cheever, A. W., Wynn, T. A., (2003). Global gene expression profiles during acute pathogen-induced pulmonary inflammation reveal divergent roles for Th1 snf Th2 responses in tissue repair, J. Immunol., 171, 3655-3667.
26. Abdrejko, K. M. Deutschman, C. S, (1997). Altered hepatic gene expression in fecal peritonitis: changes in transcription of glucogenic, beta-oxidative, and ureagenic genes, Schock, 7(3), 164-169.
27. US2006/0040301, Diagnostic assay for source of inflammation
28. Rheinart, K. (2005). The genetic response to sepsis—Can we use it to improve diagnosis?, 25th International Symposium on Intensive Care and Emergency Medicine, Brussels.
29. DE 102005013013, Verwendung von Geneaktivitats-Klassifikatoren für die in vitro Klassifizierung von Genexpressionsprofilen von Patienten mit infektiösem/nichtinfektiösem Multiorganversagen (use of gene activity classificators for in vitro classification of gene expression profiles of patients with infectious/non-infectious multiple organ failure)
30. Bone R C, Balk R A, Cerra F B, et al. (1992) The ACCP/SCCM Consensus Conference Committee (1992) Definitions for Sepsis and organ failure and guidelines for the use of innovative therapies in Sepsis. Chest 101:1656-1662; and Crit Care Med 1992; 20: 864-874.
31. Huber W, Heydebreck A, Sueltmann H, et al. (2003) Parameter estimation for the calibration and variance stabilization of microarray data. Stat. Appl. in Gen. and Mol. Biol., Vol. 2, Issue 1, Article 3

We claim:

1. A method for treatment of a local inflammation of a fever of unknown origin (FOU) in a human, comprising:
i. isolating a blood sample from a patient with a FUO;
ii. obtaining gene expression profiles from the blood sample of the patient;
wherein probes are used for obtaining the gene expression profiles;
wherein the probes comprise sequences that are specific only to the polynucleotides of SEQ-ID No. 1 to SEQ-ID No. 432 or sequences that are 80% homologous to the polynucleotides thereof;
iii. comparing the gene expression profiles of the patient to the gene expression profiles of a control group;
iv. utilizing the comparison to diagnose the local inflammation of the FUO; and
v. administering appropriate therapeutic measures to treat the FUO.

2. The method according to claim 31, wherein the probes used for obtaining the gene expression profiles further comprise three diagnostic clusters; wherein Diagnostic Cluster 1 comprises probes specific only to the polynucleotides of SEQ-ID No. 1 to SEQ-ID No. 77; wherein Diagnostic Cluster 2 comprises probes specific only to the polynucleotides of SEQ-ID No. 78 to SEQ-ID No. 191; wherein Diagnostic Cluster 3 comprises probes specific only to the polynucleotides of SEQ-ID No. 192 to SEQ-ID No. 432; wherein differential gene expression of the genes detected by Diagnostic Cluster 1 indicates the local inflammation of a FUO is peritonitis; wherein differential gene expression of the genes detected by Diagnostic Cluster 2 indicates the local inflammation of a FUO is pneumonia; and wherein differential gene expression of the genes detected by Diagnostic Cluster 3 indicates the FUO is caused by a local inflammation, but cannot distinguish between peritonitis and pneumonia.

3. The method according to claim 31, wherein gene expression profiles of at least two genes are obtained.

4. The method according to claim 31, further comprising: vi. utilizing the gene expression profiles as inclusion or exclusion criterion to decide whether patients with a FUO are included into clinical studies or excluded therefrom; and vii. utilizing the gene expression profiles to establish gene activity data for electronic further processing.

5. The method according to claim 31, further comprising at least one of: vi(a) utilizing the gene expression profile for at least one of: the production of software for the description of the individual prognosis of the patient, for diagnostic purposes, and for patient data management systems; and vi(b) utilizing the gene expression profile for at least one of: the creation of clinical expert systems and the modeling of cellular signal transduction pathways.

6. The method according to claim 31, wherein the probes further comprise sequences that are between 20 and 2000 nucleotides in length.

7. The method according to claim 31, wherein the gene expression profiles are ascertained by hybridization methods.

8. The method according to claim 37, wherein the hybridization methods comprise at least one of microarrays and real-time PCR.

9. The method of claim 32, wherein the polynucleotides are replaced with a member selected from at group consisting of synthetic analogues, aptamers, Spiegelmers, peptido- and morpholinonucleic acids.

10. A method for screening active agents against a FUO, peritonitis, and pneumonia in a human, comprising:
i. isolating a blood sample from a patient with a FUO;
ii. obtaining gene expression profiles from the blood sample of the patient;
wherein probes are used for obtaining the gene expression profiles;
wherein the probes comprise sequences that are specific only to the polynucleotides of SEQ-ID No. 1 to SEQ-ID No. 432;
wherein the probes are at least 20 nucleotides in length;
iii. comparing the gene expression profiles of the patient to the gene expression profiles of a control group;
iv. utilizing the comparison to diagnose the local inflammation of the FUO;
v. administering and active agent to the patient with a FUO;
vi. isolating a second blood sample from the patient;
vii. obtaining gene expression profiles as described in (ii); and
viii. determining efficacy of the active agent by comparing the gene expression profile obtained in (ii) to the gene expression profile obtained in (vii).

11. The method of claim 40, wherein the polynucleotides are replaced with a member selected from a group consisting of synthetic analogues, aptamers, Spiegelmers, peptido- and morpholinonucleic acids.

12. A kit for diagnosing a local inflammation of a FUO in a human diagnosed with a FUO, comprising at least two probes;
wherein the probes are used for obtaining gene expression profiles;
wherein the probes comprise sequences that are specific only to the polynucleotides of SEQ-ID No. 1 to SEQ-ID No. 432 or sequences that are 80% homologous to the polynucleotides thereof;
wherein the probes are at least 20 nucleotides in length.
